(19) United States
(12) Patent Application Publication
(10) Pub. No.: US 2019/0300844 A1
(43) Pub. Date: Oct. 3, 2019

(54) Methods for Producing Rich Cell Culture Media using Chemoautotrophic Microbes

(71) Applicant: Oakbio, Inc., Sunnyvale, CA (US)

(72) Inventors: Brian Sefton, Cupertino, CA (US); William Coleman, Redwood City, CA (US)

(21) Appl. No.: 16/443,658

(22) Filed: Jun. 17, 2019

Related U.S. Application Data
(63) Continuation-in-part of application No. 15/641,114, filed on Jul. 3, 2017.
(60) Provisional application No. 62/686,508, filed on Jun. 18, 2018.

(51) Int. Cl. C12N 1/20; C12N 1/00; C12N 1/06

(52) U.S. Cl. CPC C12N 1/20; C12N 1/06; C12N 1/005

(57) ABSTRACT

Production of nutrient-rich media, from an initial minimal medium, the rich media being suitable for cultivating heterotrophic cells, is described. These methods employ gas fermentation of photoautotrophic and/or chemoautotrophic microbes, under chemoautotrophic conditions, using carbon in common industrial waste gases to feed the growing biomass. The microbes also transform some of the carbon into organic nutrients that are released into the minimal medium thereby enriching the minimal medium. In further methods the nutrient-rich medium is then used to cultivate heterotrophic cells.

### CROSS-REFERENCE TO RELATED APPLICATION

This application claims the benefit of U.S. Provisional Patent Application No. 62/686,508 filed on Jun. 18, 2018 and is a continuation-in-part of U.S. patent application Ser. No. 15/641,114 filed on Jul. 3, 2017, both of which are incorporated herein by reference.

### BACKGROUND

### Field of the Invention

The present invention is generally related to the fields of microbial fermentation and industrial biotechnology, and more particularly to producing nutrient-rich growth media for the cultivation of heterotrophs.

### Related Art

Cell culture is the practice of growing, propagating, and maintaining cells in a liquid, or on solid or semi-solid substrate such as agar. In the practice of cell culture, the liquid or material on which the cells are cultivated is referred to as the medium. All heterotrophic cells, that is, all cells other than autotrophic cells, require a medium which comprises some form of chemical energy and carbon, and these may be provided by small molecules such as formate, acetate, and methanol, or more complex and larger molecules such as sugars and starches, and in some cases very complex and large chemicals such as proteins.

Cell culture media can have many different compositions comprising a range of components including mineral salts, sugars, amino acids, peptides, proteins, polysaccharides, hormones, growth factors and complex ingredients such as bovine serum, tryptone and yeast extract. Media compositions that contain only water and mineral salts are referred to as “minimal media.” Minimal media are inherently insufficient for heterotrophic cells as lacking some form of chemical energy and carbon. A minimal medium does not include organic compounds. Media that contain organic compounds such as sugars, yeast extract, enzymatically digested protein and other sources of energy and complex compounds are referred to as “rich media” or “complex media.” Complex or rich media are those media which essentially contain all of the required energy, carbon and other factors which the microbe(s) need to grow. Proteins are of particular importance when cultivating cell lines of multicellular organisms such as vertebrates, mollusks, and arthropods.

The following further definitions apply herein:

“Heterotrophic” is defined as meaning “requiring complex organic compounds of nitrogen and carbon (such as that obtained from yeast, plant or animal matter) for metabolic synthesis.”

“Autotrophic” is defined as meaning “requiring only carbon dioxide or carbonates (C₁ compounds) as a source of carbon and a simple inorganic nitrogen compound for metabolic synthesis of organic molecules (such as glucose).”

“Chemoautotrophic” is defined as “being autotrophic and oxidizing an inorganic compound as a source of energy.” The inorganic compound as a source of energy may include H₂ in the case of hydrogen-oxidizing bacteria, which can consume a combination of CO₂, H₂ and O₂. Examples include anaerobic acetogens that consume CO₂ for carbon and H₂ for energy. Other inorganic energy sources for chemoautotrophs may include reduced small molecules, such as H₂S, ammonium, or ferrous iron. In some instances, the carbon and energy inputs for chemoautotrophs may be combined into a single C₁ molecule. For example, carboxydotrophs and carboxydovores consume CO (carbon monoxide) for both carbon and energy, and methanotrophs consume CH₄ (methane) along with O₂ (molecular oxygen) or other oxygen-donating compounds. Chemoautotrophic metabolism is known in bacteria and archaea, and may also exist as an undiscovered trait, or as a capability conferred by genetic modification, in some other organisms. Examples of chemoautotrophs are found across numerous bacterial genera such as Cupriavidus, Rhodobacter, Methylobacterium, Methylococcus, Methylosinus, Nitrosomonas, Nitrosococcus, Nitrobacter, Nitrococcus, Paracoccus, Hydrogenothermus, Hydrogenovibrio, Clostridium, Rhodococcus, Rhodospirillum, Alcaligines, Rhodopseudomonas, and Thiobacillus, as well as in a number of genera of the archaea, including methanogens. Specific examples of chemoautotrophs include Cupriavidus necator, Cupriavidus basilensis, Rhodococcus opacus, Methylococcus capsulatus, Methylosinus trichosporium, Methylobacterium extorquens, Hydrogenothermus marinus, Rhodospirillium rubrum, Rhodopseudomonas palustrus, Paracoccus zeaxanthinifaciens, Rhodobacter sphaeroides, Rhodobacter capsulatus, and Clostridium autoethanogenum.

“Fermentation” is defined as “a metabolic process that produces chemical changes in organic substrates through the action of enzymes.” In biochemistry, it is narrowly defined as the extraction of energy from carbohydrates in the absence of oxygen. In the context of food production, it may more broadly refer to any process in which the activity of microorganisms brings about a desirable change to a foodstuff or beverage.” Even more broadly, and for the purposes of this invention, “fermentation” is a process for cultivating cells in a specialized vessel (made of glass, metal or plastic and known as a fermenter or bioreactor) under controlled process conditions in order to optimize their growth and maximize efficiency. The controlled process conditions include sterility, temperature, agitation rate, pH, input gas composition and flow rate, nutrient composition, cell density, dissolved gas concentration, biomass removal rate (for continuous or semi-continuous harvesting) and the like. Fermentation in the latter context can be aerobic or anaerobic.

“Gas fermentation” refers to a fermentation in a bioreactor wherein the metabolic processes of the chemoautotrophic cells extract energy and carbon from the gaseous inputs that are supplied to them. Gas fermentation can refer to anaerobic or aerobic process of microbe cultivation on gases. By combining these gas inputs with the simple inorganic salts in the medium, the chemoautotrophic cells convert these basic inputs into more complex biomass and other cellular products. Gas fermentation can be either aerobic or anaerobic, depending on the organism used and the feedstock gases available for fermentation. Gas fermentation is a particularly advantageous form of chemoautotrophic fermentation because the key inputs are provided by widely available and low-cost gases.

“Culturing” is defined as meaning “the act or process of cultivating living material (such as bacteria or viruses) in a prepared nutrient medium.” “Nutrient” is defined as meaning “a substance or ingredient that promotes growth, provides energy, and maintains life.” “Medium” is defined as “a nutrient system for the artificial cultivation of cells or organisms and especially bacteria.” Media can be liquid, semi-solid or solid (e.g., agar, beads or other scaffolding). Solid or semi-solid media can provide a growth support for the cells.

It should be noted that, in a typical heterotrophic fermentation, the cells are grown in media that include complex organic molecules such as sugars, amino acids, peptides, organic acids, or the like. The heterotrophic cells generally extract most of the “high-energy” forms of carbon from the medium to increase the cellular biomass, releasing the catabolized carbon as carbon dioxide, acetate, or other simple, low-value waste products. Thus, the medium that remains after the resulting biomass is harvested from the bioreactor following a heterotrophic fermentation process is usually called “spent medium,” since it generally has very low nutritional value for further cultivating heterotrophic organisms. Even when heterotrophs are selected or designed to excrete high-value products into the medium, they nevertheless must be cultivated on fairly high cost media (including such complex molecules as sugars or proteins), thus limiting the profit margin of the production process.

During chemoautotrophic fermentation, the cells similarly grow and produce biomass. However, the highly anabolic metabolism of chemoautotrophs generates an excess of nutritionally valuable products, some portion of which leaks out or is excreted into the medium.

“Higher life forms” or “higher organisms” refer to eukaryotic organisms such as yeast, fungi, microalgae, plants and animals.

A considerable expense in the commercial cultivation and maintenance of cells, particularly cultures of cells isolated from multicellular organisms, such as plants, fish, mollusks, and arthropods, is the cost of the growth medium. Such media often contain, in addition to water and various inorganic salts, a number of different peptide growth factors, amino acids, sugars, yeast extracts, protein digests of animal or vegetable origin, serums of animal origin, proteins (e.g., tryptone and peptone, or albumin such as bovine serum albumin), and other metabolites critical to the growth of the cells. A significant part of the high production cost for these media is the use of materials that are processed from animal material, such as blood and other fluids and tissues.

For example, Basal Medium Eagle (BME) is a widely used synthetic basal medium for supporting the growth of many different mammalian cells. BME contains eight B-vitamins and ten essential amino acids, plus cystine, tyrosine, and glutamine.

### BRIEF DESCRIPTION OF THE DRAWINGS

FIG. 1 is a schematic representation of an exemplary embodiment of a system, according to various embodiments of the present invention.

FIG. 2 is a schematic representation of an exemplary bioreactor, according to various embodiments of the present invention.

FIG. 3 is a flowchart representation of a method, according to various embodiments of the present invention.

FIG. 4 is a graph of experimental results showing the cultivation of Lactobacillus brevis in media produced according to an exemplary method of the present invention.

### SUMMARY

The present invention describes the production, from waste gases, of nutrient-rich media suitable for cultivating heterotrophic cells. The methods described herein use carbon in industrial waste as the bottom of a food chain that begins by gas fermenting photoautotrophic or chemoautotrophic microbes, under chemoautotrophic conditions, on that carbon and in a medium initially without organic nutrients. The microbes multiply to convert this carbon into greater biomass and under appropriate conditions can also transform some of the carbon into organic nutrients as waste byproducts that can enrich the medium so as to be suitable for cultivating heterotrophic cells further up the food chain.

An exemplary method of the invention comprises providing a minimal medium to a bioreactor, inoculating the minimal medium in the bioreactor with an inoculum including chemoautotrophic and/or photoautotrophic cells, and cultivating, chemoautotrophically, the inoculum to grow a biomass in the bioreactor by providing a gaseous input into the bioreactor until a cell density of the biomass in the medium meets a threshold, whereby the minimal medium is enriched during the cultivation to become an enriched medium. In various embodiments, the method further comprises preparing the inoculum before inoculating the minimal medium therewith. Various methods can further comprise preparing the minimal medium. Further embodiments comprise sterilizing the minimal medium and the bioreactor before inoculating the minimal medium with the inoculum. In still further embodiments the minimal medium comprises a gel. In various embodiments, minimal medium is added to the bioreactor together with a growth support for the cells.

In various embodiments, the chemoautotrophic cells or photoautotrophic cells produce a growth factor, a hormone, an antibiotic, amino acid, peptide, protein, vitamin, colorant, carotenoid, fatty acid, or oil. In various embodiments, the inoculum also includes heterotrophic cells. The inoculum also can include photoautotrophic cells, and in these embodiments the cultivating is performed in the absence of light within the bioreactor.

In various embodiments cultivating the inoculum includes adding a beneficial molecule to the enriched medium, and in some of these embodiments the beneficial molecule comprises glucose. Cultivating the inoculum includes adjusting the pH of the enriched medium, in some embodiments. The inoculum, in some instances, includes chemoautotrophic cells and the gaseous input comprises CH₄ and O₂. In some of these embodiments, the chemoautotrophic cells include cells of Methylococcus capsulatus. In various embodiments, the gaseous input comprises CO, or the gaseous input comprises CO₂ and H₂S, or the gaseous input comprises CO₂, H₂ and O₂. In still other embodiments, the inoculum includes cells of Cupriavidus necator, cells of Rhodobacter capsulatus, or cells of both. The inoculum can include cells of a carboxydotroph or cells of Rhodococcus opacus, in various embodiments.

Various embodiments of the invention further comprise destroying cells in the enriched medium so as to release their contents into the enriched medium, destroying cells can include lysing the cells, in some instances. In various embodiments the enriched medium includes one or more of a growth factor, a hormone, an antibiotic, an amino acid, a peptide, a protein, a vitamin, a colorant, a carotenoid, a fatty acid, or an oil.

In various embodiments the method further comprises separating the insoluble biomass from the enriched medium, such as by centrifugation, filtration, or gravity-based separation. In some of these embodiments, the enriched medium, after separation, includes at least 1 gram of D-glucose per liter. In some embodiments, the method further comprises after separation, one or more of adding mineral salts to the enriched medium, adjusting the pH of the enriched medium, filtering, the enriched medium, providing an enzymatic treatment to the enriched medium, performing a chromatographic separation upon the enriched medium, or performing a selective precipitation from the enriched medium. In methods that separate insoluble biomass from enriched medium, some methods further comprise removing ammonium ions from the enriched medium.

In various embodiments, the method further comprises cultivating cells of a heterotroph in the enriched medium separated from the biomass, thereby depleting the enriched medium. In some of these methods, the heterotroph includes a yeast, fungus, algae, archaeon, bacterium, or mammal. The heterotroph cells are derived from a cell line of a multicellular aquatic organism, in further embodiments.

Further, the present invention is directed to various enriched media produced by the methods described herein.

### DETAILED DESCRIPTION

The present invention describes the production, from waste gases, of nutrient-rich media suitable for cultivating heterotrophic cells. Production of such media includes at least cultivating chemoautotrophic and/or photoautotrophic cells chemoautotrophically via gas fermentation in an initially minimal medium, and then after sufficient cultivation harvesting that enriched medium. The enriched medium can then be used for the cultivation of heterotrophs such as yeast, fungi, microalgae, plants and animals. The cells cultivated in the bioreactor may comprise a single species or single strain of a chemoautotrophic or photoautotrophic microbe, or they may comprise multiple strains or species. In addition, these cells may be co-cultured with one or more species or strains of various selected heterotrophic microbes, the purpose of which is to supply additional desired nutritional components (probiotics, etc.) to the medium that are not produced by the chemoautotrophic or photoautotrophic cells alone. A co-culture or consortium with heterotrophic cells can be designed so that the heterotrophic cells provide more added value to the final product than they consume. In some embodiments, the intracellular contents of the resulting biomass can be added to the enriched medium.

As used herein, “first medium” is the initial medium used to supply basic nutrients for an initial round of fermentation. As used herein, “second medium” is a supernatant enriched medium, resulting from the enrichment of the first medium, that remains after separation from all or most of the biomass. The second medium is therefore a rich medium. During chemoautotrophic fermentation, the cells grow and increase biomass, but their highly anabolic metabolism also generates an excess of nutritionally valuable products, some portion of which leaks out or is excreted into the growth medium. Media remaining after enrichment from chemoautotrophic fermentation is referred to as “second media” in order to distinguish it from the nutritionally inferior “spent media” resulting from heterotrophic fermentation. After the biomass has been largely or wholly removed, the second medium contains many complex substances suitable for supporting the growth of heterotrophic cells. This second medium can therefore be collected during or after the fermentation, processed to remove any remaining cells or cell debris (if desired), and re-used as a nutritionally advantageous growth medium or additive for the cultivation of heterotrophic cells. Also used herein, “enriched medium” refers to the first medium after gas fermentation has begun and broadly encompasses both that medium during cultivation as well as the second medium after a separation process.

Chemoautotrophic and photoautotrophic microbes cultivated on industrial waste gases can be a particularly rich and profitable source of nutrients, since these microbes must produce all of their cellular constituents (including sugars, fatty acids, carotenoids, cofactors, vitamins, peptides and proteins) de novo from simple, and generally inexpensive inputs (e.g., hydrogen, carbon dioxide, oxygen, water and mineral salts). This chemoautotrophic mode of production also has the advantage that vitamins and proteins can be synthesized at lower cost than by fermentation of heterotrophic microbes on sugar, for example.

Second media produced according to the present invention can contain similar or identical components as compared to prior art rich media, and can provide equivalent nutritive value and therefore can supplement or entirely replace animal-derived and other expensive ingredients for various cell culture applications. In some cases, this not only reduces production costs, but also provides sources of culture media that do not require killing or harming animals. In other cases, components available in the media may provide benefits not found in known rich media and therefore the product of the process is itself novel over the prior art. The medium can consist entirely of liquid, or it can be formulated into a gel (using agar, for example), or contain solid or semi-solid material (such as beads) for use as a growth support for the cells.

It is important to note that typical prior art growth media for heterotrophs have initial compositions designed to be depleted as the growing cells are cultivated. According to the present invention, the fermentation of the chemoautotrophic cells begins in a minimal medium. The input of the feedstock gases, combined with the chemoautotrophic growth of the cells that feed on the mineral salts and the feedstock gases—as well as the growth of any additional heterotrophic cells that might be included as part of a consortium and that feed off of products from the chemoautotrophic growth—actually builds up the nutritional quality of the enriched medium as the cultivation proceeds.

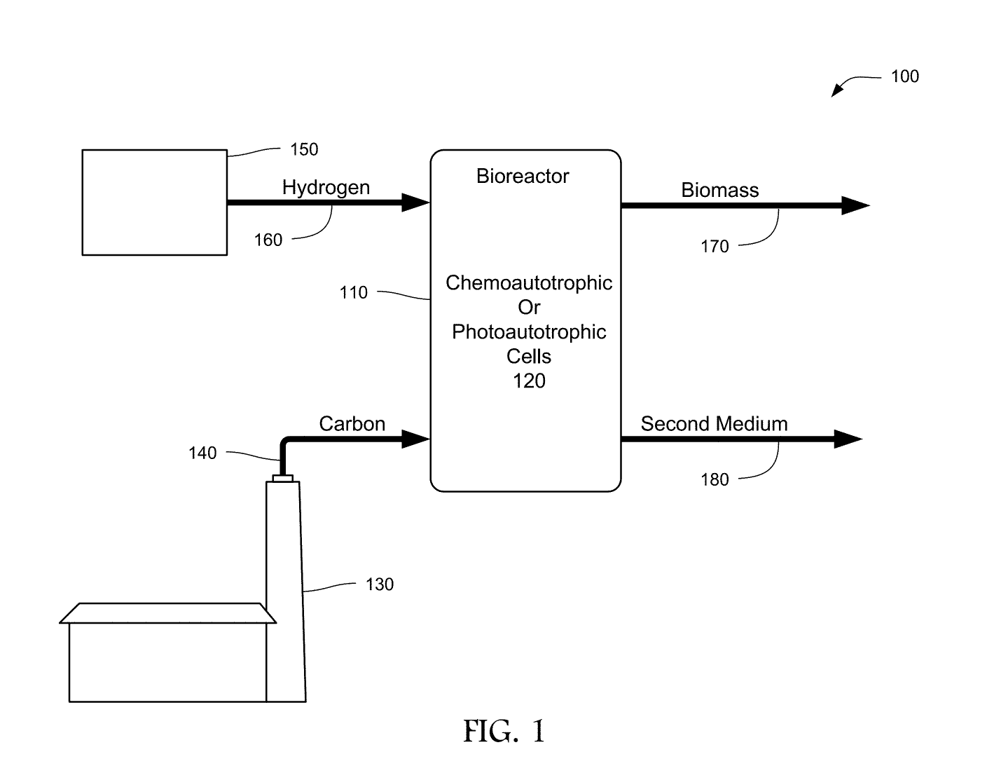

FIG. 1 shows a schematic representation of an exemplary system 100 of the invention. The system 100 comprises a bioreactor 110 including photoautotrophic and/or chemoautotrophic cells 120. The system 100 also comprises a source of carbon 130, such as an industrial source that produces a waste stream 140 including one or more of the carbon oxides, carbon monoxide and carbon dioxide. Examples of sources of carbon 130 include cement manufacturing facilities, power plants that burn fossil fuels, ferrous metal products manufacturing (e.g., casting and forging), non-ferrous products manufacturing, foodstuffs manufacturing, fermentation plants which produce ethanol or other bioproduction manufacturing, gasification of biomass, gasification of coal, and chemical manufacturing such as petroleum refining, carbon black production, ammonia production, methanol production and coke manufacturing.

The system 100 further comprises an optional source of molecular hydrogen 150, such as a hydrogen storage tank, hydrogen pipeline, steam methane reformer, gasifier or an electrolysis system. The hydrogen source 150 produces a hydrogen stream 160 including molecular hydrogen as a source of energy for the chemoautotrophic or photoautotrophic cells grown chemoautotrophically, that is, in the absence of light. In various embodiments of the invention, the cells in the bioreactor being grown chemoautotrophically derive both carbon and energy from one waste stream 140, such as methane or carbon monoxide, and in those embodiments the source of molecular hydrogen 150 is not necessary. In further embodiments, a waste stream 130 includes a source of carbon and also a source of hydrogen such as might be produced by a gasifier. FIG. 1 also schematically illustrates that the two output streams of the system 100, after removal from the bioreactor 110 and separation, are accumulated biomass 170 and an enriched or “second” medium 180. In some embodiments, the biomass 170 is gasified (not shown) and the output is used as a second carbon source 130.

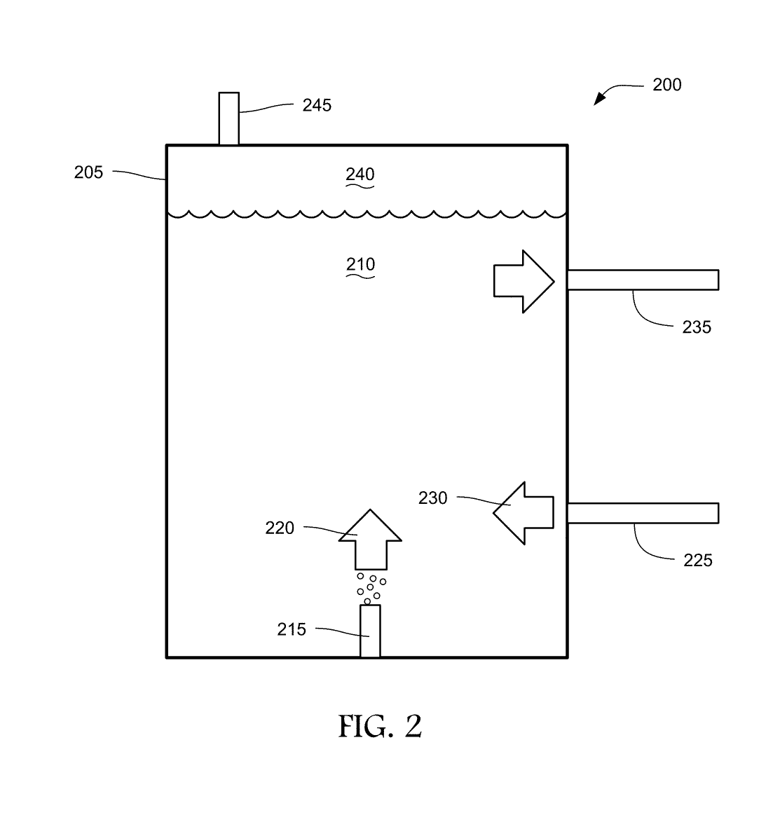

FIG. 2 shows a schematic representation of a bioreactor 200 as one example of a suitable bioreactor for methods of the present invention. Bioreactor 200 can comprise either a synthesis vessel for production of cells to inoculate the first medium and for use in conjunction with a separate growth vessel for the production of biomass in an enriched medium, or bioreactor 200 can comprise a vessel suitable for both of the synthesis and growth stages. In FIG. 2, bioreactor 200 includes a vessel 205 that in operation holds a quantity of a liquid medium 210 containing the chemoautotrophic or photoautotrophic cells and optionally other heterotrophic cells in culture. The bioreactor 200 also includes an input port 215 through which gas 220 can be introduced into the vessel 205 for introduction into the medium 210, a media inlet port 225 through which fresh media 230 can be introduced into the vessel 205, and a media outlet port 235 through which the medium 210 can be removed, for example, to separate enriched medium from the insoluble biomass. The bioreactor 200 can also comprise a headspace 240 and a gas release valve 245 to vent gases from the headspace 240. In some embodiments, the gas release valve 245 is attached to a recirculation system to return vented gases back to the input port 215, and may include a manifold (not shown) through which to make additions to optimize the gas composition.

In various embodiments, the bioreactor 200 can be a continuously stirred tank reactor, a loop bioreactor, or any other design appropriate for gas fermentation. The bioreactor 200 can further include controlled agitation for mixing, various probes for measuring pH, dissolved gases, and culture density, and controls for the gases, temperature regulation, and the like. Agents for controlling foaming can also be added to the bioreactor 200.

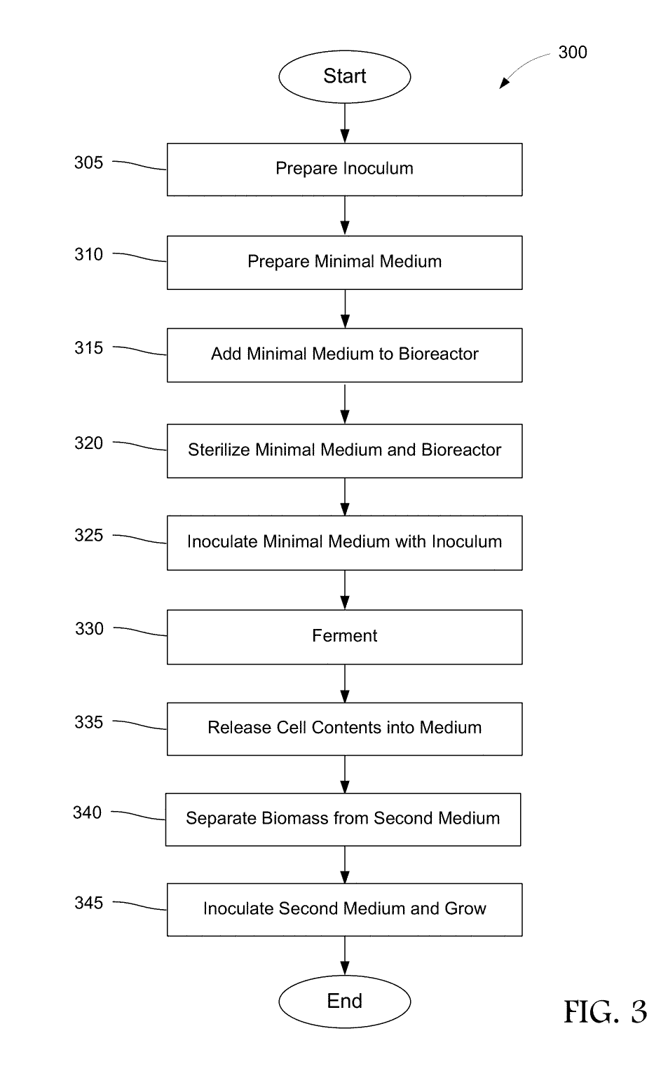

FIG. 3 illustrates an exemplary method 300 of the present invention. While some steps are noted as optional, steps not noted as optional are not necessarily essential. The method 300 comprises an optional step 305 of preparing an inoculum and an optional step 310 of preparing a minimal medium. The method 300 then comprises a step 315 of adding the minimal medium to a bioreactor and an optional step 320 of sterilizing the minimal medium and the bioreactor. The method 300 then comprises a step 325 of inoculating the sterile minimal medium with the inoculum, a step 330 of fermenting, until the cell density meets a threshold, by feeding a gas into the bioreactor. The method 300 then comprises an optional step 335 of releasing the contents of the cells into the enriched medium, and then an optional step 340 of separating the biomass from the second medium. In an optional step 345 the second medium is inoculated with heterotrophic cells of the type that the second medium was designed to support.

In the step 305 an inoculum is prepared, enough to inoculate a bioreactor such as bioreactor 200. The step is optional is as much as certain embodiments of the method 300 can begin with a pre-made inoculum. The inoculum includes cells of at least one species of a photoautotrophic or chemoautotrophic microbe, and can be prepared using either a rich medium or a minimal medium together with a gas feedstock. The particular species of microbe or microbes chosen for the inoculum, and any other heterotrophic microbes included therein, are selected to yield a suitable second medium that is tailored for the benefit of later cultivating some particular higher organism in the second medium, such as a bacterium, an archaebacterium, a microalgae, a fungus, a mold, or a yeast. Preparing the inoculum can include co-culturing chemoautotrophic, photoautotrophic, and/or heterotrophic cells together in the same medium or one or more strains in separate media. In the latter case, preparing the inoculum can include preparing amounts of chemoautotrophic, photoautotrophic, and/or heterotrophic cells at different times and storing those amounts until all are ready for use.

In some embodiments, the chemoautotrophic microbe comprises a single chemoautotrophic cell strain, such as Cupriavidus necator or Methylococcus capsulatus, or Rhodobacter capsulatus, or Rhodococcus opacus, or a chemoautotroph that has been genetically modified to produce a beneficial heterologous product or products. Chemoautotrophic strains can be natural, contain mutations, be genetically modified, or contain one or more genes edited via CRISPR, in order to produce a valuable small molecule, growth factor, a hormone, an antibiotic, amino acid, peptide, sugar, polysaccharide, protein, vitamin, colorant, carotenoid, fatty acid, organic acid, nucleic acid, oil, glycolate, hydrocarbon, polyhydroxyalkanoate, phasin, gene transfer agent (GTA) or other biomolecules that can then be utilized by non-autotrophic microbes and other heterotrophic cells as growth substrates and growth regulators. Examples of photoautotrophic microbes which might be used this way include Rhodospirillium rubrum, Rhodopseudomonas palustrus, Paracoccus zeaxanthinifaciens, Rhodobacter sphaeroides, Rhodobacter capsulatus, cyanobacteria such as spirulina and anabaena.

Deposit of Biological Material

The following microbes have been deposited with the American Type Culture Collection, 10801 University Boulevard, Manassas, Va. 20110-2209, USA (ATCC):

TABLE 1

| Microbe Designation | ATCC No. | Deposit Date |
|---|---|---|
| Rhodobacter capsulatus OB-213 | PTA-12049 | Aug. 25, 2011 |

This deposit was made under the provisions of the Budapest Treaty on the International Recognition of the Deposit of Microorganisms for the Purpose of Patent Procedure and the Regulations thereunder (Budapest Treaty). This assures maintenance of viable cultures for 30 years from the date of the deposit. The organisms will be made available by ATCC under the terms of the Budapest Treaty, and subject to an agreement between Oakbio, Inc. and ATCC, which assures permanent and unrestricted availability of the progeny of the cultures to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 USC § 122 and the Commissioner's rules pursuant thereto (including 37 CFR § 1.12 with particular reference to 886 OG 638).

The assignee of the present application has agreed that if the cultures on deposit should die or be lost or destroyed when cultivated under suitable conditions, they will be promptly replaced on notification with a viable specimen of the same culture. Availability of the deposited strain is not to be construed as a license to practice the invention in contravention of the rights granted under the authority of any government in accordance with its patent laws.

In the step 310 a minimal medium including mineral salts in water is prepared. The step is also optional is as much as certain embodiments of the method 300 can begin with a pre-made minimal medium. Examples of minimal media for chemoautotrophs include Repaske's medium for hydrogenotrophs and NMS medium for methanotrophs. Recipes for these exemplary minimal media are publicly available such as through the American Type Culture Collection (ATCC).

In a step 315 the minimal medium is added to a bioreactor and in a step 320 the minimal medium and the bioreactor are sterilized. The minimal medium can be sterilized, for example, via heat, radiation or by passing the minimal medium through a sterile filter (e.g., a 0.2 um filter apparatus). In step 315 the minimal medium can comprise a gel. Also in step 315 the minimal medium can be added to the bioreactor together with a growth support, such as beads.

In a step 325 the sterile medium in the bioreactor is inoculated with an inoculum, such as that prepared in step 305. In some embodiments, inoculating the bioreactor with the inoculum includes sequentially introducing separately prepared quantities of different cells.

In a step 330 the inoculum is fermented, to cultivate it into a biomass, by feeding a gaseous feedstock comprising one or more of CO₂, CO, CH₄, H₂, H₂S, O₂, N₂, or NH₃ into the bioreactor. Where more than one gas is included in the feedstock, the several gases are supplied in an appropriate combination and proportion for the species of cells being cultivated. Particular examples include mixtures of CO₂, H₂, and O₂, mixtures of CH₄ and O₂, and mixtures of CO₂ and H₂S. The fermentation is maintained until the cells of the biomass achieve a threshold density, typically greater than 0.5 grams cell dry weight per liter (CDW/L), but preferably greater than about 2 grams CDW/L. During step 330 beneficial molecules are secreted into the minimal medium by the cells of the growing biomass to create an enriched medium. In further embodiments, during step 330, additional beneficial molecules can be added to the enriched medium to further increase the nutritional quality thereof. For instance, in order to create a culture medium suitable for mammalian cells, glucose can be added to increase the concentration of glucose to an acceptable level for rapid growth. In other cases, addition of iron or other minerals may be required. Likewise, the pH can be adjusted by adding acid or base, and additional mineral salts, yeast extract, tryptone, phenol red or other components can be included.

In optional step 335 the medium may be sterilized or treated to kill, lyse, or otherwise inactivate or destroy the cells in the enriched medium in such a way as to release their contents so that the enriched medium further contains the intracellular amino acids, proteins, nucleic acids, polyhydroxyalkanoates, organic acids and other factors which render the medium more useful for culturing cells of higher organisms.

In a step 340 the second medium is harvested. In some embodiments this is achieved by a separation process such as centrifugation, filtration, or gravity-based separation, for example, to remove the biomass. In some embodiments, this processing may include sterile filtration through a 0.2 um filter membrane, so that the resulting liquid does not contain any microbial cells. In some embodiments, the second medium harvested in step 340 can be modified by the addition of mineral salts, adjustment of pH, filtration, enzymatic treatment, chromatographic separation, selective precipitation, and/or other operations to render the second medium more useful for culturing cells of higher organisms. For example, it might be advantageous to selectively remove ammonium ions from the second medium, such as with a wash step, if the heterotrophic fermentation will be inhibited by high concentrations of this component. The same is true for lactate. In some embodiments, the second medium contains cells of the chemoautotrophic organisms, or others, from the original inoculum.

Second media produced by the chemoautotrophic methods described herein contain over 1 gram of D-glucose per liter, as well as significant amounts of vitamin B2, vitamin B3, vitamin B12, biotin, pantothenate, glutamate, methionine and peptides starting from first media containing no glucose, vitamins, amino acids or proteins at all. Heterotrophic bacteria, yeast (Phaffia, Saccharomyces), fungus (Aspergillus), and bacteria (Lactobacillus, Bacillus, Bifidobacterium, Brevundimonas, Escherichia) can be cultivated in an unmodified second medium produced by cultivating chemoautotrophic bacteria. Second media of the invention can also be used as a foundation for, or a supplement to, more complex media preparations for higher organisms.

In an optional step 345 the second medium is inoculated with cells of a higher life form. The cells are cultivated in the second medium until the cells are harvested, or until the entire culture is harvested, or until some component thereof (e.g., a recombinant protein) is isolated from the resulting medium. These microbes may include bacteria, archaea, microalgae, fungi, molds, yeasts or others. Examples of such microbes include:

Ascomycota, Aspergillus niger, Aspergillus oryzae, Bacillus coagulans, Bacillus lentus, Bacillus licheniformis, Bacillus megaterium, Bacillus pumilus, Bacillus subtilis, Bacteroides amylophilus, Bacteroides capillosus, Bacteroides ruminocola, Bacteroides suis, Basidiomycota, Bifidobacterium adolescentis, Bifidobacterium animalis, Bifidobacterium bifidum, Bifidobacterium infantis, Bifidobacterium lactis, Bifidobacterium longum, Bifidobacterium thermophilum, Bifidobacterum breve, Saccharomyces cerevisiae, Blakeslea trispora, Pichia pastoris, Kluyveromyces lactis, Hansenula polymorpha, Lactobacillus acidophilus, Lactobacillus brevis, Lactobacillus bulgaricus, Lactobacillus casei, Lactobacillus cellobiosus, Lactobacillus curvatus, Lactobacillus delbruekii, Lactobacillus fermentum, Lactobacillus helveticus, Lactobacillus johnsonii, Lactobacillus lactis, Lactobacillus paracasei, Lactobacillus parafarraginis, Lactobacillus plantarum, Lactobacillus reuterii, Lactobacillus rhamnosus, Lactobacillus salivarius, Lactobacillus sporogenes, Lactococcus lactis, Leuconostoc mesenteroides, Pediococcus acidilactici, Pediococcus cerevisiae, Pediococcus pentosaceus, Propionibacterium shermanii, Propionibacterium freudenreichii, Saccharomyces boulardii, Streptococcus cremoris, Streptococcus diacetylactis, Streptococcus faecium, Streptococcus intermedius, Streptococcus lactis, and Streptococcus thermophiles.

### Example 1: Production of the Second Medium Using Gas Fermentation of Chemoautotrophic Bacteria

In this first example, a gas fermentation was carried out using a New Brunswick BioFlo 4500 30L continuously stirred tank bioreactor, modified for gas fermentation. An inoculum of chemoautotrophic and heterotrophic species was prepared with Cupriavidus necator, Rhodobacter capsulatus, Rhodospirillum rubrum, Rhodobacter sphaeroides, Rhodopseudomonas palustris as the chemoautotrophic species and Bacillus megaterium, Lactobacillus acidophilus, Lactobacillus casei subspecies casei as the heterotrophic (probiotic) species.

To prepare the inoculum, bacterial cultures were cultured from frozen stocks inoculated into 15 ml of sterile Luria Bertani broth and grown in a temperature-controlled incubator shaking at 200 rpm and at 30 C overnight, or until the cultures reached an absorbance at 620 nm of 0.6. Absorbance Units (au). These were further cultured in a chemoautotrophic growth medium, containing, per liter, the following:

| | | |
|---|---|---|
| Phosphate (PO₄) from 10x solution | 100 | mL |
| Ammonium chloride (NH₄Cl) from 10x solution | 200 | ml |
| Sodium bicarbonate (NaHCO₃) from 20 g/200 mL solution | 2 | ml |
| Nickel from 100 mM (NH₄)2Ni(SO₄)2.6H₂O solution4 | 0.166 | ml |
| Distilled H2O (diH2O) | 674 | ml |

After the medium was sterilized the following sterile mineral salts were added:

| | | |
|---|---|---|
| Trace Elements from Schlegel Solution ‘E’ | 2 | mL |
| CaCl₂•2H₂O from 200 g/L solution5 | 0.1 | ml |
| MgSO₄•7H₂O from 100x solution6 | 10 | ml |
| FeSO₄•7H₂0 from 0.1 g/100 mL solution | 12 | ml |

Many of the above ingredients were added from stock solutions of much higher concentration. The phosphate 10× stock was prepared from 40 g of sodium phosphate dibasic (Na₂HPO₄) anhydrous and 66.7 g of potassium phosphate monobasic (KH₂PO₄) anhydrous mixed in 1 L of diH₂0. The ammonium chloride 10× stock was prepared from 18 g of NH₄Cl mixed in 1 L of diH₂0. The Sodium bicarbonate stock solution was prepared from 20 g of NaHCO₃ mixed in 200 mL of diH₂0. Nickel can be alternatively provided by 100 μL of 100 mM NiCl₂. The calcium chloride can be provided from 200 g of CaCl2.H20 in 1 L of diH₂O; a 10,000× concentrated stock solution. Lastly, the magnesium sulfate can be prepared by adding the appropriate amount from a solution containing 113.05 g of MgSO4.7H2O; a 100× concentrated stock solution [help me understand this].

The bioreactor was filled with ˜20 L of the medium, then sterilized for 30 minutes using the bioreactor's onboard sterilization cycle, cooled to room temperature, and then the remaining mineral salts were added. The various components of the consortial inocula were then added through a sterile port.

Hydrogen gas was supplied to the bioreactor by a 9 kW Proton S40 electrolyzer supplied with ultra-pure water. O₂ and CO₂ were supplied from compressed gas cylinders fitted with gas regulators to lower the pressure to about 20 psi. Gas mixing was controlled by a set of three mass flow controllers according to the ratio 80:10:10 (H₂:CO₂:O₂). The gas flow rate into the bioreactor increased from 1-8 SLPM as fermentation progressed. The gas head pressure within the bioreactor was 10 psi. A temperature of 30 C, a pH of 6.8, and an impeller agitation rate of 300 rpm were maintained.

The bioreactor was operated in a semi-continuous harvesting mode for 32 days. Every 24 hours, 10 L of the reactor contents were removed via a sterile port, and the same volume of sterile fresh medium was added back to the bioreactor. Bacterial biomass was separated from the enriched medium by centrifugation and then lyophilized for later use. Aliquots of the remaining supernatant medium were sterilized by filtration through a sterile, disposable 0.2 um filtration apparatus and frozen at −20 C resulting in the “second medium.”

Frozen samples of the second medium from Day 11 and Day 23 of the fermentation were subjected to a spent medium analysis, with the following results:

TABLE 2
Analysis of second medium from chemoautotrophic
H₂:CO₂:O₂ gas fermentation

| | | |
|---|---|---|
| ANALYTE | DAY 11 | DAY 23 |

| | | | | |
|---|---|---|---|---|
| Ammonium | 63.83 | mmol/L | 107.03 | mmol/L |
| Glucose | 1.44 | g/L | 1.22 | g/L |
| Lactate | 1.04 | g/L | 0.82 | g/L |
| Glutamate | 0.014 | g/L | 0.049 | g/L |
| Methionine | 0.060 | g/L | 0.052 | g/L |

| | | |
|---|---|---|
| Thiamine (B1) | ND | ND |

| | | | | |
|---|---|---|---|---|
| Riboflavin (B2) | 1.01 | mg/L | 0.60 | mg/L |
| Nicotinic Acid (B3) | 2.04 | mg/L | 1.79 | mg/L |
| Niacinamide (B3) | 1.36 | mg/L | 1.19 | mg/L |
| Ca Pantothenate (B5) | 2.13 | mg/L | 1.73 | mg/L |

| | | |
|---|---|---|
| Pyridoxine (B6) | ND | ND |

| | | | | |
|---|---|---|---|---|
| Biotin (B7) | 1.53 | mg/L | 1.70 | mg/L |

| | | |
|---|---|---|
| Folic Acid (B9) | ND | ND |

| | | | | |
|---|---|---|---|---|
| Cyanocobalamine (B12) | 0.97 | mg/L | 0.93 | mg/L |

ND = Below the limit of detection
Amino acids not listed were below the limits of detection.

A BCA protein assay (Pierce) indicated that the Day 11 sample also contained 0.83 g/L protein. Analysis of the protein molecular weight by SDS-PAGE with Coomassie Blue staining (Invitrogen) indicated that most of the protein consisted of small molecular weight peptides of less than about 10 kD (not shown).

Bacterial biomass from each sample was separated from the second medium by centrifugation and lyophilized for later use (approximately 8 g CDW for each liter harvested). The second medium, now cell free was collected and stored at 4 C.

### Example 2: Heterotrophic Cultivation of Lactobacillus brevis on Second Medium from Day 11 of the Gas Fermentation

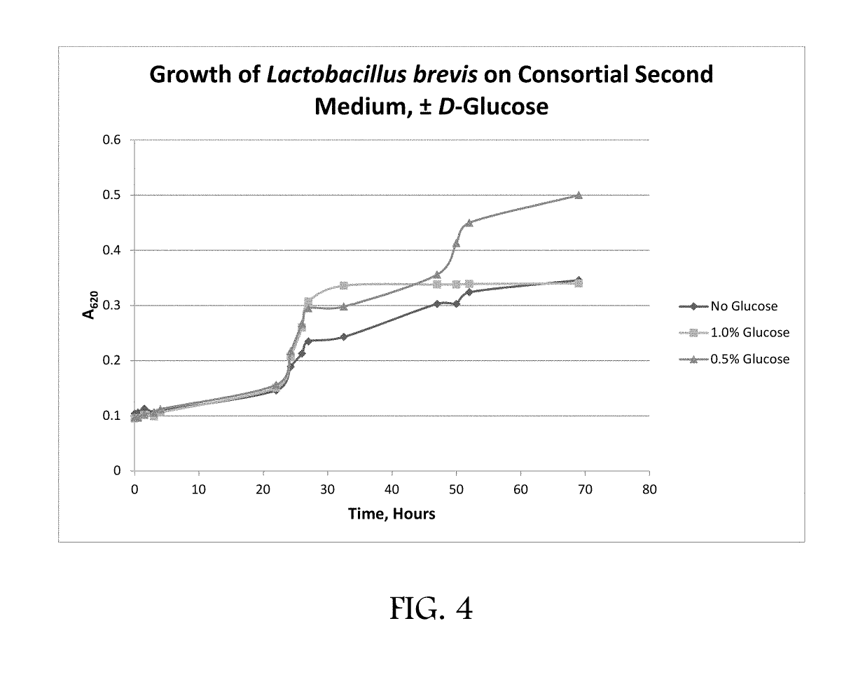

Cells of a frozen stock of the heterotroph Lactobacillus brevis were inoculated in a 1:50 ratio into 30 ml of sterile second medium from Day 11 of the fermentation described in Example 1. This inoculum, in 250 ml baffled culture flasks, was shaken at 100 rpm at 28 C on a rotary shaker in a temperature-controlled incubator. One flask received no additions (“No Glucose”), and the other two flasks contained the same second medium as the first flask but with additional 0.5% (w/v) glucose and 1.0% (w/v) glucose, respectively (above the level of 0.14% that was already present in the second medium). Samples were removed at various times, and the optical density at 620 nm (A620) of 200 uL aliquots was measured in a microplate reader. The growth curves are shown in FIG. 4.

The results of FIG. 4 show that the heterotrophic bacteria more than tripled their density on the second medium without any glucose supplementation. Additional glucose stimulated their growth above this level, particularly at the later stage for the 0.5% addition, although 1% glucose may be less effective as the culture ages. This demonstrates that the second medium can be used to cultivate heterotrophic cells as either a complete medium or as a significant medium component. This method also makes it possible to effectively cultivate heterotrophic cells indirectly on gas, and thereby take advantage of feedstocks and culture conditions for which they are not metabolically suited. It also makes it possible to extract additional value from the products of the gas fermentation over and above the original extracted biomass.

In the foregoing specification, the invention is described with reference to specific embodiments thereof, but those skilled in the art will recognize that the invention is not limited thereto. Various features and aspects of the above-described invention may be used individually or jointly. Further, the invention can be utilized in any number of environments and applications beyond those described herein without departing from the broader spirit and scope of the specification. The specification and drawings are, accordingly, to be regarded as illustrative rather than restrictive. It will be recognized that the terms “comprising,” “including,” and “having,” as used herein, are specifically intended to be read as open-ended terms of art.

1. A method comprising:
providing a minimal medium to a bioreactor;
inoculating the minimal medium in the bioreactor with an inoculum including chemoautotrophic and/or photoautotrophic cells; and
cultivating, chemoautotrophically, the inoculum to grow a biomass in the bioreactor by providing a gaseous input into the bioreactor until a cell density of the biomass in the medium meets a threshold, whereby the minimal medium is enriched during the cultivation to become an enriched medium.

2. The method of claim 1 wherein the chemoautotrophic cells or photoautotrophic cells produce a growth factor, a hormone, an antibiotic, amino acid, peptide, protein, vitamin, colorant, carotenoid, fatty acid, or oil.

3. The method of claim 1 wherein the inoculum also includes heterotrophic cells.

4. The method of claim 3 wherein the inoculum includes photoautotrophic cells and the cultivating is performed in the absence of light within the bioreactor.

5. The method of claim 1 wherein cultivating the inoculum includes adding a beneficial molecule to the enriched medium.

6. The method of claim 5 wherein the beneficial molecule comprises glucose.

7. The method of claim 1 wherein cultivating the inoculum includes adjusting the pH of the enriched medium.

8. The method of claim 1 wherein the inoculum includes chemoautotrophic cells and the gaseous input comprises CH₄ and O₂.

9. The method of claim 8 wherein the chemoautotrophic cells include cells of Methylococcus capsulatus.

10. The method of claim 1 wherein the gaseous input comprises CO.

11. The method of claim 1 wherein the gaseous input comprises CO₂ and H₂S.

12. The method of claim 1 wherein the gaseous input comprises CO₂, H₂ and O₂.

13. The method of claim 1, wherein the inoculum includes cells of Cupriavidus necator, cells of Rhodobacter capsulatus, or cells of both.

14. The method of claim 1, wherein the inoculum includes cells of a carboxydotroph.

15. The method of claim 1, wherein the inoculum includes cells of Rhodococcus opacus.

16. The method of claim 1 further comprising preparing the inoculum before inoculating the minimal medium therewith.

17. The method of claim 1 further comprising preparing the minimal medium.

18. The method of claim 1 further comprising sterilizing the minimal medium and the bioreactor before inoculating the minimal medium with the inoculum.

19. The method of claim 1 further comprising destroying cells in the enriched medium so as to release their contents into the enriched medium.

20. The method of claim 19 wherein destroying cells includes lysing the cells.

21. The method of claim 1 wherein the enriched medium includes one or more of a growth factor, a hormone, an antibiotic, an amino acid, a peptide, a protein, a vitamin, a colorant, a carotenoid, a fatty acid, or an oil.

22. The method of claim 1 further comprising separating the insoluble biomass from the enriched medium.

23. The method of claim 22 wherein the enriched medium, after separation, includes at least 1 gram of D-glucose per liter.

24. The method of claim 22 further comprising, after separation, one or more of adding mineral salts to the enriched medium, adjusting the pH of the enriched medium, filtering the enriched medium, providing an enzymatic treatment to the enriched medium, performing a chromatographic separation upon the enriched medium, or performing a selective precipitation from the enriched medium.

25. The method of claim 22 wherein separating the biomass from the enriched medium includes centrifugation, filtration, or gravity-based separation.

26. The method of claim 22 further comprising removing ammonium ions from the enriched medium.

27. The method of claim 22 further comprising cultivating cells of a heterotroph in the enriched medium separated from the biomass, thereby depleting the enriched medium.

28. The method of claim 27 wherein the heterotroph includes a yeast, fungus, algae, archaeon, bacterium, or mammal.

29. The method of claim 27 wherein the heterotroph cells are derived from a cell line of a multicellular aquatic organism.

30. The method of claim 1 wherein the minimal medium comprises a gel.

31. The method of claim 1 wherein the minimal medium is added to the bioreactor together with a growth support for the cells.

32. An enriched medium produced by the method of claim 1.
